(19) United States
(12) Patent Application Publication
(10) Pub. No.: US 2021/0228081 A1
(43) Pub. Date: Jul. 29, 2021

(54) METHOD AND APPARATUS FOR DETERMINING WETNESS PERCEPTION

(71) Applicants: Sandhya Kumar, Tallahassee, FL (US); Surabhi Kumar, Tallahassee, FL (US); Amrita Kumar, Tallahassee, FL (US)

(72) Inventors: Sandhya Kumar, Tallahassee, FL (US); Surabhi Kumar, Tallahassee, FL (US); Amrita Kumar, Tallahassee, FL (US)

(21) Appl. No.: 17/111,239

(22) Filed: Dec. 3, 2020

Related U.S. Application Data
(60) Provisional application No. 62/965,132, filed on Jan. 23, 2020.

(51) Int. Cl. A61B 5/00

(52) U.S. Cl. CPC A61B 5/0053; A61B 5/4058; A61B 2560/0214; A61B 5/48; A61B 2562/0247; A61B 5/4029

(57) ABSTRACT

Processes, scales, and devices to measure and quantify wetness perception in humans. Exemplary devices and scales utilize sensor fusion of temperature and pressure modalities, for which humans have dedicated receptors in the skin, to understand how the perception of wetness comes about. Processes test the utility of wetness perception as a biomarker for assaying peripheral neuropathy. Wetness perception devices include a Peltier module. The temperature of the Peltier module can be varied precisely using a computer-aided feedback system, mounted on a load scale to enable concomitant pressure measurements. Devices may include an insulation chamber with desiccators in place to lower internal humidity and prevent condensation. Wetness perception can be used as a non-invasive biomarker for disease-related peripheral neuropathy in which sensory mechanisms are disrupted.

### PRIORITY

This application claims priority to, and the benefit of, U.S. Provisional Patent Application No. 62/965,132, filed on Jan. 23, 2020 and titled “DESIGN AND DEVELOPMENT OF A WETNESS PERCEPTION MONITOR AND A PROCESS TO MEASURE THE PERCEPTION OF WETNESS IN HUMANS,” the disclosure of which is hereby incorporated by reference herein in its entirety.

### BACKGROUND

The present disclosure is directed to wetness perception and more particularly, to systems and methods for determining wetness perception.

### SUMMARY

Humans have no receptors to sense wetness, yet the ability to distinguish dry from wet is routine. The perception of wetness may emerge from the sensory fusion of information regarding pressure and temperature.

For humans, the perception of wetness is important for maintaining homeostasis and adaptation to surroundings by thermoregulatory processes. For example, the amount of sweat produced is partially dependent on the saturation of water on the surface of the skin.

The skin is the largest organ of the human body and has a variety of sensors that transduce multi-modal information relating touch, vibration, pressure and temperature into an electrical impulse-mediated internal representation for processing by the brain. The Pacinian corpuscles, for example, are mechano-receptors, responsible for sensitivity to deep pressure touch and high frequency vibration. The Meissner's corpuscles (or tactile corpuscles), located just beneath the epidermis, are mechanoreceptors responsible for sensitivity to light touch. These are distributed throughout the skin, but concentrated in areas especially sensitive to light touch, such as the fingertips, palms, soles, lips, tongue, and face. Merkel nerve endings are mechanoreceptors found in the skin and mucosa of vertebrates that provide touch information to the brain. The strategic placement of these bio-sensors within the skin-syncytium and their organization into networks is perhaps the single most important factor underlying differential sensitivity to stimuli at various locations throughout the body. These biosensors transduce different aspects of the stimulus (temperature versus pressure, for instance) that is synthesized in the brain. These mechano-receptors are responsible for detecting pressure and temperature on the surface of the skin and sending a signal to the brain. However, humans possess no specific receptors to sense wetness. Because of this, the perception of wetness emerges from the sensor fusion of information regarding pressure and temperature being detected by humans.

Large populations of people, approximately 25%-30% of Americans, including 8% of Americans who are over the age of 65 (Cleveland Clinic—Neuropathy), suffer from peripheral neuropathy, for which there is no definitive diagnostic tool available. Early intervention might allow physicians to take steps to prevent neuropathy. A few conditions that lead to peripheral neuropathy include diabetes, cancer-related chemotherapy, inflammatory infections, protein abnormalities and heredity disorders. According to the Mayo Clinic, peripheral neuropathy can be identified through an extensive neurological exam. Currently, there is no simple noninvasive test to definitively diagnose peripheral neuropathy.

In some embodiments, systems and methods are disclosed for determining and quantifying the perception of wetness in human subjects, and using the conditions under which this perception occurs as a biomarker for diagnosis of conditions such as peripheral neuropathy.

In some embodiments, the wetness perception device disclosed utilizes sensor fusion to understand the perception of wetness and its use as a potential biomarker for assaying peripheral neuropathy.

In some embodiments, the wetness perception device quantifies the perception of wetness in humans by determining the range of temperatures and pressures at which human subjects perceive wetness. It is designed to provide a relatively moisture-free environment in which a subject's sensation of ambient temperature and pressure can be accurately assessed.

In some embodiments, the wetness perception device is an insulated and moisture resistant chamber that has an opening, a thermal element, and a pressure sensor. In some embodiments, the opening is sized to accept a portion of the human subject for positioning proximate to a portion of the chamber. In some embodiments, the thermal element is positioned within the chamber and configured to maintain at least the portion of the chamber at a predetermined temperature. In some embodiments, the pressure sensor is positioned within the chamber and configured to determine a pressure applied to the portion of the human subject.

In some embodiments, the thermal element within the wetness perception device contains a test surface. In some embodiments, the thermal element, and therefore the test surface, is placed on the pressure sensor. This enables the portion of the human subject positioned within the opening of the chamber to be placed on the test surface of the thermal element, which is placed on the pressure sensor. Further, this allows for varying the temperature at the test surface that the portion of the human subject is placed on, while measuring pressure exerted on the test surface by the portion of the human subject.

In some embodiments, because the perception of wetness arises from combined assessments of pressure and temperature, the wetness perception device and the data collected from it are used to determine whether the disruption of either of these sensory modalities would disrupt the perception of wetness. Thus, in some embodiments, wetness perception can be used as a non-invasive biomarker for disease-related peripheral neuropathy in which those sensory mechanisms are disrupted. Further, in some embodiments, wetness perception can be used as a non-invasive biomarker for any condition involving nerve damage, for example, Diabetes, Human Immunodeficiency Virus (HIV), Celiac Disease, Amyloidosis, Fabry's disease, Alcoholism, autoimmune conditions such as Lupus and Vasculitis, cancers such as Lymphoma or Myeloma, cancer-related chemotherapy, or Lyme Disease.

### BRIEF DESCRIPTION OF THE DRAWINGS

The present disclosure, in accordance with one or more various embodiments, is described with reference to the following figures. The drawings are provided for purposes of illustration only and merely depict typical or example embodiments. These drawings are provided to facilitate an understanding of the concepts disclosed herein and do not limit the breadth, scope, or applicability of these concepts. It should be noted that for clarity and ease of illustration these drawings are not necessarily made to scale.

FIG. 1 is a block diagram of a system for detecting wetness perception in a human subject, in accordance with some embodiments of the present disclosure;

FIG. 2 is a block diagram illustrating further details of elements of a system for detecting wetness perception in a human subject, in accordance with some embodiments of the present disclosure;

FIG. 3 is a block diagram illustrating further details of elements of a system for detecting wetness perception in a human subject, in accordance with some embodiments of the present disclosure;

FIG. 4 conceptually illustrates operation of a system for detecting wetness perception in a human subject, in accordance with some embodiments of the present disclosure;

FIG. 5 shows a flow diagram of an illustrative process for detecting wetness perception in a human subject, in accordance with some embodiments of the present disclosure;

FIG. 6 shows scatterplots depicting correlation of the temperature at which wetness is perceived with pressure and participant's age, in accordance with some embodiments of the present disclosure;

FIG. 7 shows graphs depicting wetness perception as a function of age, in accordance with some embodiments of the present disclosure;

FIG. 8 shows graphs depicting wetness perception in humans varies with gender (male/female), in accordance with some embodiments of the present disclosure;

FIG. 9 shows graphs depicting compromise of wetness and dampness perception in human subjects with deficits in sensory perception, in accordance with some embodiments of the present disclosure;

FIG. 10 shows a graph depicting exemplary average temperature ranges of wetness perception as discovered through methods and systems disclosed herein, in accordance with some embodiments of the present disclosure.

### DETAILED DESCRIPTION OF EMBODIMENTS OF THE DISCLOSURE

Unless otherwise defined herein, scientific and technical terms used in this application shall have the meanings that are commonly understood by those of ordinary skill in the art. In case of conflict, the present specification, will control.

The practice of the present disclosure will employ, unless otherwise indicated, suitable techniques of detecting wetness perception in a human subject.

Throughout this specification and embodiments, the word “comprise,” or variations such as “comprises” or “comprising,” will be understood to allow the inclusion of a stated integer or group of integers, but not the exclusion of any other integer or group of integers. “Comprising” may be synonymous with “including” or “containing. ”

The term “including” is used to mean “including, but not limited to. ” “Including” and “including but not limited to” are used interchangeably.

Any example(s) following the term “e.g.” or “for example” is not meant to be exhaustive or limiting.

Unless otherwise required by context, singular terms shall include pluralities and plural terms shall include the singular.

The articles “a”, “an” and “the” are used herein to refer to one or to more than one (i.e., to at least one) of the grammatical object of the article. By way of example, “an element” means one element or more than one element. As used herein, the term “about” modifying the quantity of an ingredient, parameter, calculation, or measurement in the compositions of the disclosure or employed in the methods of the disclosure refers to variation in the numerical quantity that can occur, for example, through typical measuring and/or liquid handling procedures used for making isolated polypeptides or pharmaceutical compositions in the real world; through inadvertent error in these procedures; through differences in the manufacture, source, or purity of the ingredients employed to make the compositions or carry out the methods; and the like without having a substantial effect on the chemical or physical attributes of the compositions or methods of the disclosure. Such variations can be within an order of magnitude, typically within 10%, more typically still within 5%, of a given value or range. The term “about” also encompasses amounts that differ due to different equilibrium conditions for a composition resulting from a particular initial mixture. Whether or not modified by the term “about”, the paragraphs include equivalents to the quantities. Reference to “about” a value or parameter herein also includes (and describes) embodiments that are directed to that value or parameter per se. For example, description referring to “about X” includes the description of “X.” Numeric ranges are inclusive of the numbers defining the range.

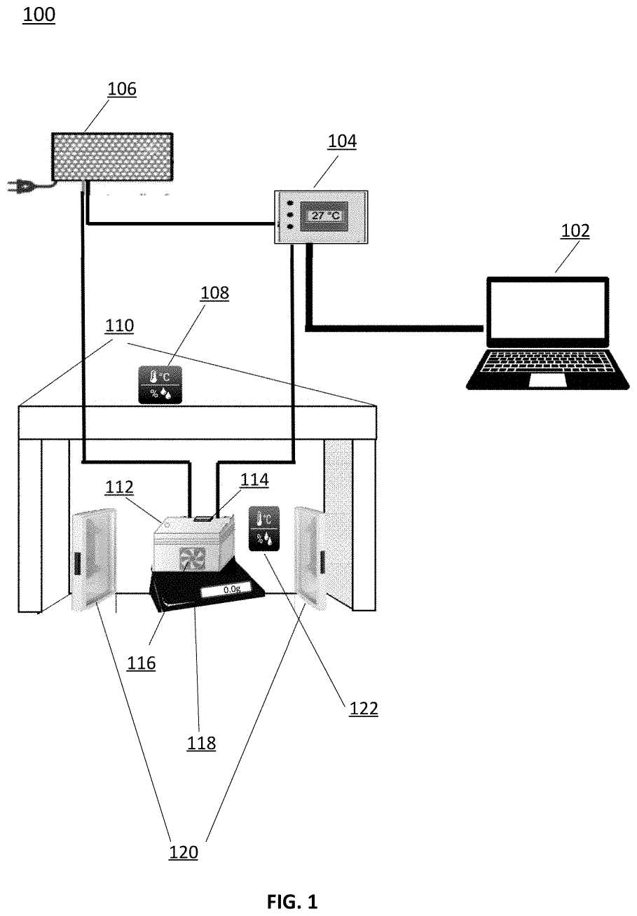

FIG. 1 shows an illustrative example of a system 100 for detecting wetness perception in a human subject, in accordance with some embodiments of the present disclosure. System 100 includes computer 102, temperature controller 104, power supply 106, outside hygrometer/thermometer module (hygrometer/thermometer module mounted outside the test chamber) 108, test chamber 110, Peltier module 112, thermocouple 114, cooling fan 116, weigh scale 118, two desiccators 120, and inside hygrometer/thermometer module (hygrometer/thermometer module mounted inside the test chamber) 122. The computer 102 may be any computing device capable of controlling the temperature controller 104. The temperature controller 104 can be any computer-interfacing temperature controller, such as a thermoelectric temperature controller configured to instruct the maintenance of a constant desired temperature. The power supply 106 can be any power supply, in some embodiments, it may be a DC power supply. The outside hygrometer/thermometer module 108 can be any device or combination of devices that measures ambient humidity and temperature. The test chamber 110 can be any chamber that is thermally insulated, airtight excepting the small opening sized to accept a portion of a human subject, and sized to accommodate any desired portion of a human test subject and any instrumentation therein. The Peltier module 112 is a solid-state active heat pump that transfers heat with consumption of electrical energy, depending on the direction of the current. A Peltier module can also be referred to as a Peltier device, Peltier heat pump, solid state refrigerator, or thermoelectric cooler (TEC), although embodiments of the disclosure contemplate use of any thermal element or device for maintaining a specified temperature at a surface. Any thermoelectric generator may be used in place of a Peltier module, including a Seebeck generator. The thermocouple 114 can be any device that communicates temperature information to the temperature controller 104. The cooling fan 116 can be any device that draws cooler air from outside the test chamber 110 and expels warm air from inside the test chamber 110. The weigh scale 118 can be any well-balanced scale for measuring load. In some embodiments, the weigh scale 118 may be a digital scale, although any suitable weighing mechanism may be employed. The desiccators 120 are used to further limit the amount of moisture within the device and prevent condensation on the test surface of the Peltier module. Any device that limits moisture and prevents condensation can be used in place of the desiccators. The inside hygrometer/thermometer module 122 can be any device or combination of devices that measures ambient humidity and temperature. In one embodiment, the test chamber 110 contains two desiccators 120 on either side of the Peltier module 112, which is placed atop the weighing scale 118, although any number of desiccators 120 may be employed at any locations within test chamber 110. The Peltier module 112 is controlled by the temperature controller 104, which supplies a current to the Peltier module 112 that is proportional to the difference between a set temperature specified by the user through the computer 102 and the current temperature on the surface of the Peltier module 112 sensed by the thermocouple 114. The cooling fan 116 located at the bottom of the Peltier module helps dissipate the heat generated by the Peltier effect. The power supply 106 powers the temperature controller 104 and the cooling fan 116. The inside hygrometer/thermometer module 122 and the outside hygrometer/thermometer module 108 measure differences in humidity and temperature inside and outside of the test chamber 110.

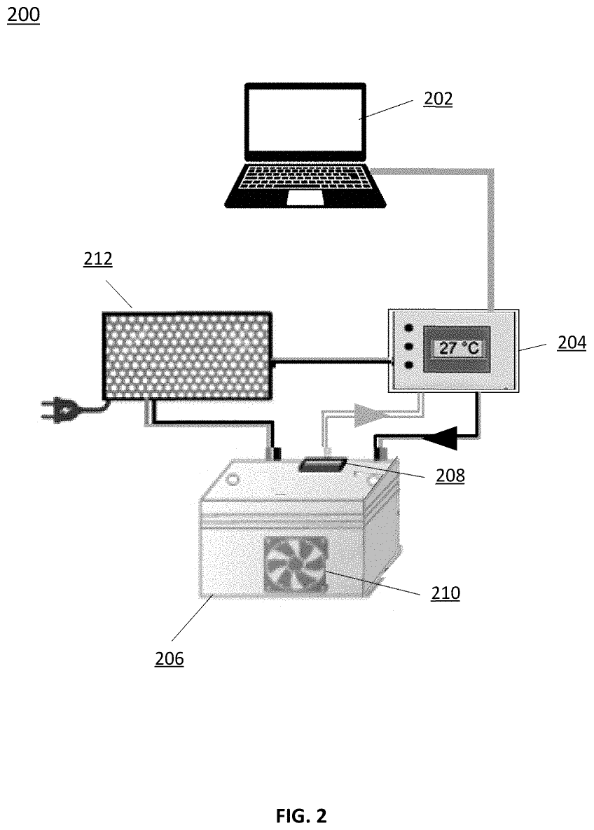

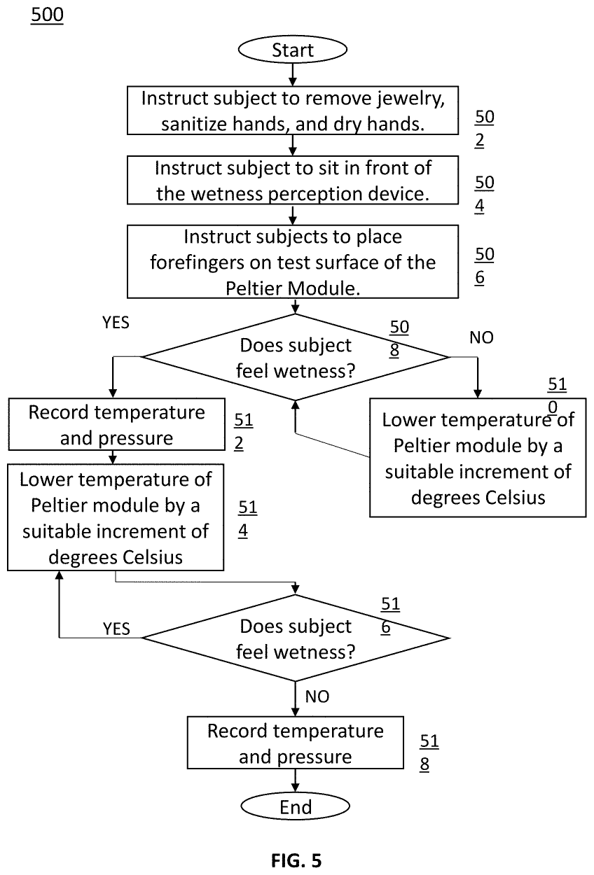

FIG. 2 shows an illustrative block diagram of system 200 for maintaining a specific temperature at a specific surface, for the purpose of detecting wetness perception in a human subject, in accordance with some embodiments of the present disclosure. System 200 includes computer 202, temperature controller 204, Peltier module 206 with thermocouple 208 and cooling fan 210, and power supply 212. System 200 illustrates selected elements of system 100 in order to depict how the system maintains a specified temperature at the surface of the Peltier module 206 while maintaining a desired environment. The Peltier module 206 is controlled by the temperature controller 204, which supplies a current to the Peltier module 206, proportional to the difference between a set temperature specified by the user through the computer 202 and the current temperature on the surface of the Peltier module 206, sensed by the thermocouple 208. The human subject is subjected to predetermined temperatures on the surface of the Peltier module 206, and those temperatures are stepwise varied (described further in FIG. 5). The cooling fan 210 is positioned to help dissipate heat generated by the Peltier module 206. The power supply 212 powers the temperature controller 204 and the cooling fan 210.

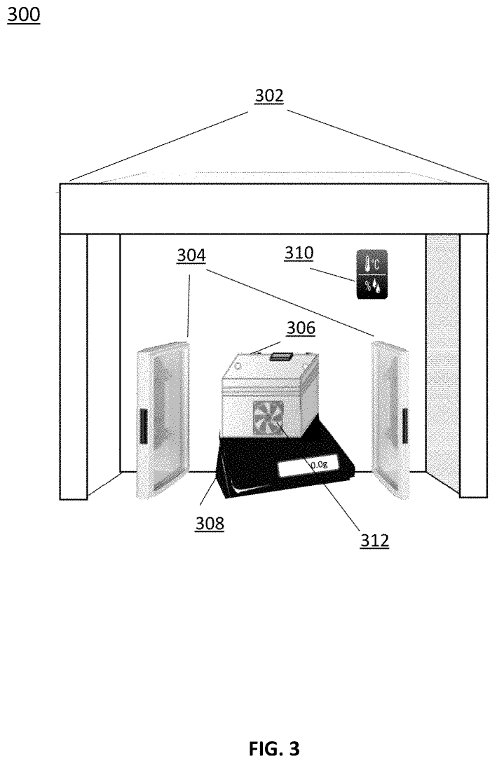

FIG. 3 shows an illustrative example of system 300 for maintaining a constant environment within an enclosed space, for the purpose of detecting wetness perception in a human subject, in accordance with some embodiments of the present disclosure. System 300 includes test chamber 302, desiccators 304, Peltier module 306, weighing scale 308, inside hygrometer/thermometer module (hygrometer/thermometer module mounted inside the test chamber) 310, and cooling fan 312. System 300 depicts mechanical operation of the controlled environment inside the test chamber 302. The test chamber 302 houses the two desiccators 304, which limit moisture and prevent condensation within the chamber, on either side of the Peltier module 306 which is placed atop the weighing scale 308. The test chamber also houses the inside hygrometer/thermometer module 310 to measure the humidity and temperature within the test chamber, and cooling fan 312 to dissipate heat that may be generated by the Peltier effect. Minimizing moisture, preventing condensation, measuring to maintain a regular humidity level and regular temperature, and dissipating heat all contribute to creating a reduced-moisture environment within the test chamber 302, preventing excess environmental moisture from interfering with the perception of wetness in test subjects and generating spurious moisture perception results.

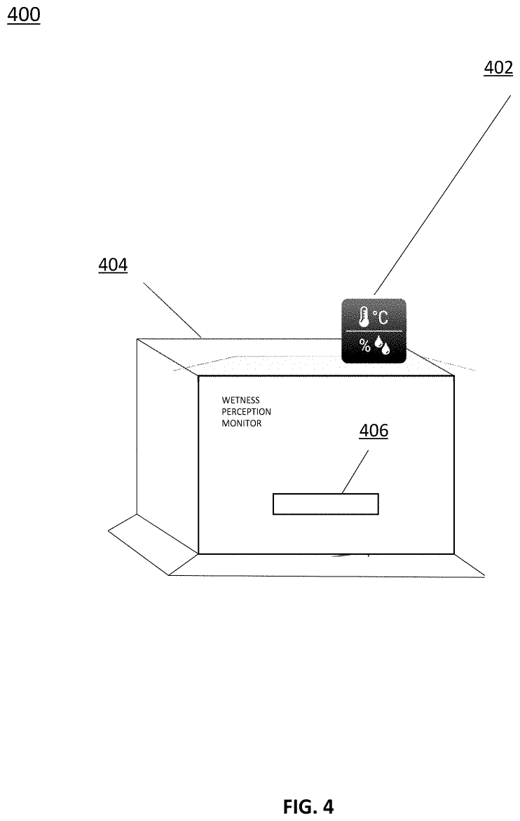

FIG. 4 shows an illustrative example of system 400 for depicting an enclosed environment with the exception of an opening sized to accept a portion of a human subject, for the purpose of detecting wetness perception in a human subject in accordance with some embodiments of the present disclosure. System 400 includes outside hygrometer/thermometer module (hygrometer/thermometer module mounted outside the test chamber) 402, test chamber 404, and chamber opening 406. System 400 is a simplified version of system 100, depicting the outside view of the test chamber 404, in order to emphasize the mostly enclosed design of the test chamber 404. The outside hygrometer/thermometer module 402 measures the humidity and temperature outside the test chamber 404. The test chamber 404 is enclosed excepting the chamber opening 406 in order to maintain a specific, controlled environment within the test chamber 404. In this embodiment, the chamber opening 406 at the front of the test chamber is sized to allow for the subject to place their palm into the chamber and upon the Peltier module 112.

FIG. 5 shows a flow diagram of an illustrative process 500 for detecting wetness perception in a human subject, in accordance with some embodiments of the present disclosure. At 502, the subject is instructed to remove jewelry, sanitize hands, and dry hands. At 504, the subject is instructed to sit in front of the wetness perception device. At 506, the subject is instructed to place their forefingers (or other body parts) onto the test surface of the Peltier module, which is at a set starting temperature. In some embodiments, the starting temperature may be 25° C. At 508, the subject is asked if they feel wetness. If No at 508, the process moves to 510. At 510, the temperature of the Peltier module is lowered by a suitable increment of degrees Celsius. In some embodiments, the increment may be 0.1 degrees Celsius. The process then returns to 508. If Yes at 508, process moves to 512. At 512, the temperature and pressure are recorded. At 514, the temperature of the Peltier module is lowered by a suitable increment of degrees Celsius. In some embodiments, the increment may be 0.1 degrees Celsius. At 516, the subject is asked if they feel wetness. If yes at 516, the process returns to 514. If no at 516, the process moves to 518. At 518, the temperature and pressure are recorded. In some embodiments, the process may be executed only once on the subject's dominant hand. In some embodiments, the process may be repeated multiple times for each of the subject's hands. Execution of this process thus yields the temperatures and pressures at which subjects begin to perceive wetness on their forefingers (or other body parts).

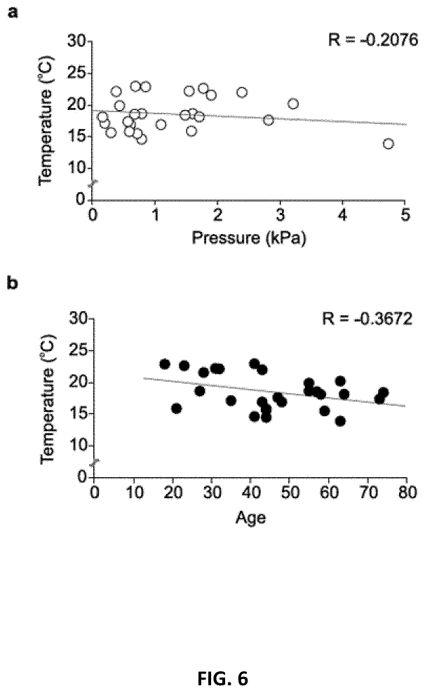

FIG. 6 shows scatterplots depicting the correlation of the temperature at which wetness is perceived with pressure (a) and participant's age (b), as determined by systems of embodiments of the disclosure. This data is fitted with linear regression lines (gray) that show a weak negative correlation for both.

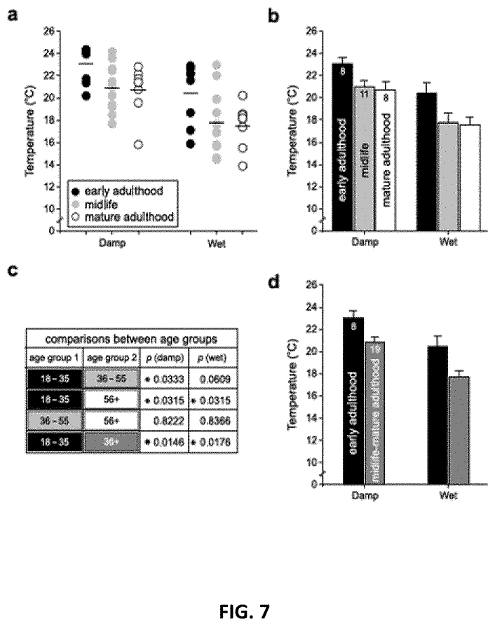

FIG. 7 shows graphs depicting how wetness perception in humans varies with age, as determined by systems of embodiments of the disclosure. Raw data (a, bar indicates mean) and histograms (b-c) of the average temperatures at which dampness (damp) and wetness (wet) are perceived by human subjects are segregated according to the indicated developmental stages (corresponding to color matched age-groups specified in panel d). Data within bar plots indicate the number of subjects (n) and error bars indicate standard error of the mean. Age-grouping of participants (based on their reported age and color-coded according to their developmental stage identified in the histograms) and statistical comparison of the differences in averaged wetness and dampness perception (p values) for participants in the various age groups are shown in a histogram (d). FIG. 7 thus shows that the average temperature that human subjects in early adulthood begin to perceive dampness is approximately 23° C. and wetness is approximately 20° C., whereas the average temperature that midlife to mature adults begin to perceive dampness is approximately 21° C. and wetness is approximately 17° C.

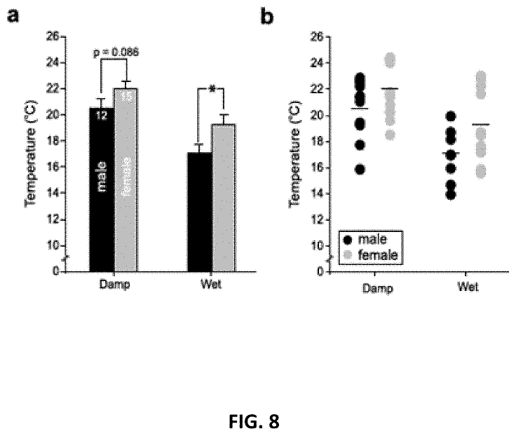

FIG. 8 shows graphs depicting how wetness perception in humans varies with gender (male/female), as determined by systems of embodiments of the disclosure. Histograms (a) and raw data (b, bar indicates mean) show the average temperatures at which dampness (damp) and wetness (wet) are perceived by human subjects, segregated according to gender. Data within bar plots indicate number of subjects (n) and error bars indicate standard error of the mean (* p<0.05, t-test). FIG. 8 thus shows that the average temperature that male human subjects begin to perceive dampness is approximately 20° C. and wetness is approximately 17° C., and the average temperature that female human subjects begin to perceive dampness is approximately 22° C. and wetness is approximately 19° C.

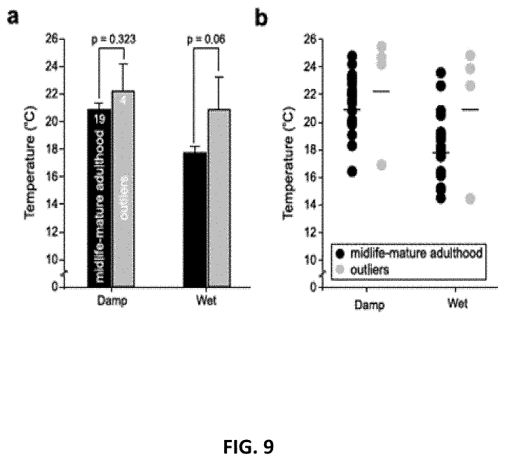

FIG. 9 shows graphs depicting how wetness and dampness perception may be compromised in human subjects with deficits in sensory perception, as determined by systems of embodiments of the disclosure. The histograms (a) and the raw data (b, bar indicates mean) show the average temperatures at which dampness (damp) and wetness (wet) are perceived by mid-life to mature adult human subjects and the outliers (including both high and low) identified within this data set. The difference in the perception of wetness between these groups of participants is nearly statistically significant. The data within the bar plots indicate number of subjects (n), and the error bars indicate standard error of the mean. FIG. 9 thus shows that the average temperature that human subjects in midlife to mature adulthood begin to perceive dampness is approximately 21° C. and wetness is approximately 17° C., and the average temperature that the outliers within the data set perceive dampness is 22° C. and wetness is approximately 20° C.

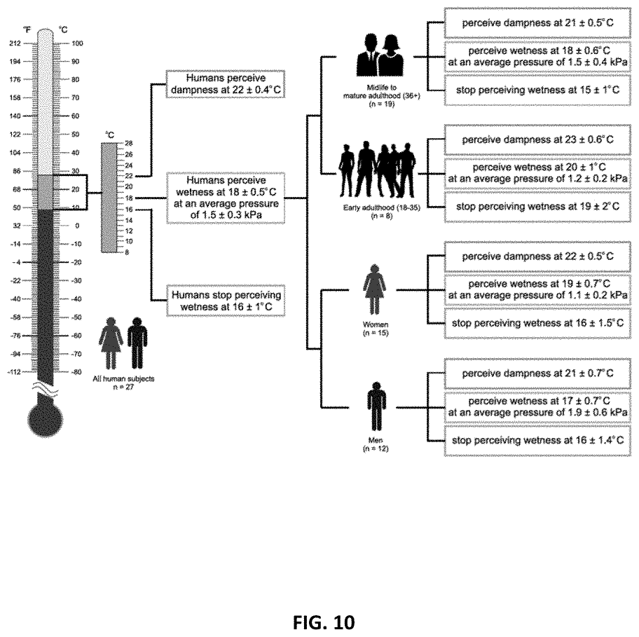

FIG. 10 shows a graph depicting exemplary average temperature ranges of wetness perception as discovered through methods and systems disclosed herein. In some embodiments, human subjects perceive wetness at an average temperature of 22±0.4° C. and the sensation of wetness is extinguished at an average temperature of 16±1° C., with measurements being made at an average tactile pressure of 1.5±0.3 kPa. In some embodiments, young adults (18-35) may begin to sense wetness at significantly higher temperatures (23.05±0.59° C.) than middle-aged adults (36-55) or mature adults (56+), who may begin to sense wetness at similar temperatures (20.95±0.64° C. and 20.72±0.78° C. respectively). In some embodiments, women may begin to sense wetness at higher average temperatures (22.06±0.52° C.) than men (20.55±0.68° C.). In some embodiments, temperatures and pressures at which human subjects begin to sense wetness are uncorrelated, suggesting that they are processed independently at the sensory level. In some embodiments, subjects whose wetness perception readings deviate from the averages tend to have self-reported deficits in sensory perception.

In some embodiments, wetness perception can be used as a biomarker to detect neuropathy when the temperature range of wetness perception for a subject deviates from the average temperature range of wetness perception. In some embodiments, a midlife to mature adult subject with nerve damage or a neuropathic condition may begin to sense wetness at a temperature closer to 22.22±1.99° C., whereas the average temperature that midlife to mature adults begin to sense dampness and wetness is 20.85±0.48° C. Thus, for example, a likelihood of nerve damage or the presence of a neuropathic condition may be deemed to be detected if the temperature at which a subject begins to perceive wetness is significantly higher than the average temperature at which a subject in their demographic begins to perceive wetness. Embodiments of the disclosure may thus be employed to detect any condition that may affect wetness or dampness perception.

The foregoing is merely illustrative of the principles of this disclosure and its various embodiments. Various modifications may be made by those skilled in the art without departing from the scope of this disclosure. The above-described embodiments are presented for purposes of illustration and not of limitation. The present disclosure also can take many forms other than those explicitly described herein. Accordingly, it is emphasized that this disclosure is not limited to the explicitly disclosed methods, systems, and apparatuses, but is intended to include variations and modifications thereof, which are within the spirit of the following claims.

What is claimed is:

1. A method for detecting wetness perception in a human subject, the method comprising:
determining a perception of wetness by a human subject, according to a temperature and a pressure applied to the human subject.

2. The method of claim 1, wherein the determining further comprises varying the temperature applied to the subject while measuring the pressure applied to the subject.

3. The method of claim 1, wherein the determining further comprises determining a perception of dampness by the human subject, according to the temperature and the pressure applied to the human subject.

4. The method of claim 1, wherein the determining further comprises applying the temperature using one or more Peltier devices.

5. The method of claim 1, wherein the determining further comprises using a thermally sealed enclosure sized to accept a portion of the human subject.

6. The method of claim 1, wherein the determining further comprises determining the perception of wetness while the temperature and the pressure are applied to the human subject.

7. The method of claim 1, wherein the determining further comprises determining a perception of wetness at a portion of the human subject having the temperature and the pressure applied thereto and desiccating an atmosphere proximate to the portion of the human subject.

8. The method of claim 1, further comprising repeating the determining for differing ones of the human subjects so as to determine differing perceptions of wetness and determining a wetness perception scale from the differing perceptions of wetness.

9. The method of claim 8, wherein the wetness perception scale comprises average temperatures and pressures at which wetness is perceived as a function of at least one of age, gender, or disease status.

10. The method of claim 1, further comprising diagnosing a disease or a medical condition of the human subject according to the determined perception of wetness.

11. The method of claim 10, wherein the disease or medical condition of the human subject further comprises one or more of a peripheral neuropathy or a central nervous system disorder of the human subject according to the determined perception of wetness.

12. An apparatus for measuring wetness perception in a human subject, the apparatus comprising:
an insulated and moisture resistant chamber having an opening sized to accept a portion of the human subject for positioning proximate to a portion of the chamber;
a thermal element positioned within the chamber and configured to maintain at least the portion of the chamber at a predetermined temperature; and
a pressure sensor positioned within the chamber and configured to determine a pressure applied to the portion of the human subject.

13. The apparatus of claim 12, wherein the insulated and moisture resistant chamber further comprises a thermally sealed enclosure to minimize internal humidity.

14. The apparatus of claim 12, wherein the insulated and moisture resistant chamber further comprises foam lining and duct tape insulation around the opening and throughout to minimize fluctuations in temperature and humidity.

15. The apparatus of claim 12, wherein the thermally sealed enclosure further comprises a plurality of desiccators located on either side of the thermal element and pressure sensor to prevent condensation.

16. The apparatus of claim 12, wherein the thermal element further comprises a plurality of Peltier devices housed within the thermally sealed enclosure.

17. The apparatus of claim 12, wherein the thermal element further comprises a feedback system to precisely control the temperature of the Peltier devices.

18. The apparatus of claim 17, wherein the feedback system is powered by a power supply.

19. The apparatus of claim 12, wherein the thermal element further comprises hygrometer modules to measure differences in humidity and temperature inside and outside of the chamber.

20. The apparatus of claim 12, wherein the thermal element further comprises a cooling fan located at the bottom of the Peltier module to dissipate the heat generated by the Peltier effect.

21. The apparatus of claim 20, wherein the cooling fan is powered by a power supply.

22. The apparatus of claim 12, wherein the pressure sensor further comprises a weighing scale.
